(19) United States
(12) Patent Application Publication
(10) Pub. No.: US 2005/0175646 A1
(43) Pub. Date: Aug. 11, 2005

(54) Cosmetic composition that mimics sebum and the use thereof

(76) Inventors: Philippe Catroux, Nogent-sur-Marne (FR); Nelly Billoni, Valmondois (FR); Jose Cotovio, Dammartin en Goele (FR); Bruno Buan, Bagnolet (FR); Gilles Rubinstenn, Paris (FR)

Correspondence Address: BURNS DOANE SWECKER & MATHIS L L P, POST OFFICE BOX 1404, ALEXANDRIA, VA, 22313-1404, US

(21) Appl. No.: 10/509,760

(22) PCT Filed: Oct. 28, 2002
(86) PCT No.: PCT/FR02/03696

(30) Foreign Application Priority Data
Oct. 30, 2001 (FR) 01/14033

(51) Int. Cl. A61K 7/00

(52) U.S. Cl. 424/401

(57) ABSTRACT

The invention relates to: a cosmetic composition that mimics sebum; a preparation method thereof; the use of same in cosmetics; the use of same for the preparation of a skin equivalent comprising a sebum equivalent; a skin equivalent comprising a sebum equivalent; and the use of a skin equivalent comprising a sebum equivalent.

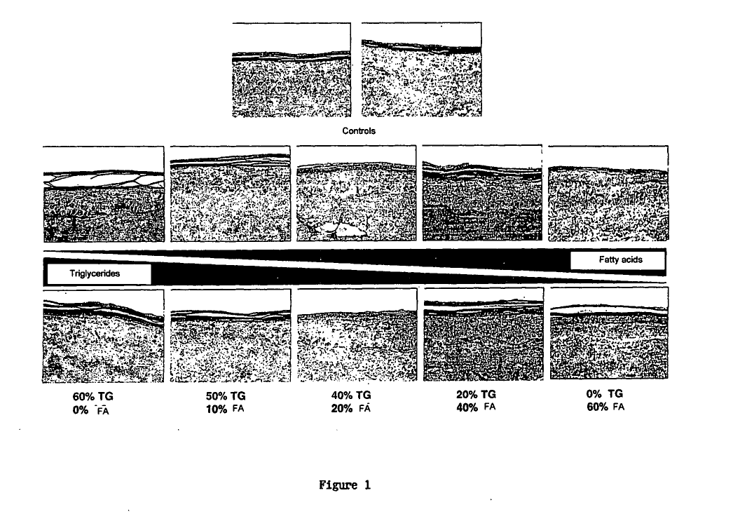

The subject of the invention is a cosmetic composition that mimics sebum, a preparation method, the use in cosmetics of a cosmetic composition that mimics sebum, the use of a cosmetic composition that mimics sebum for the preparation of a skin equivalent comprising a sebum equivalent, a skin equivalent comprising a sebum equivalent and the use of a skin equivalent comprising a sebum equivalent.

Sebum is a product excreted by the sebaceous glands of the skin of most mammals. The excretion mechanism is a holocrine mechanism by which sebum is deposited on the surface of the skin. One of the roles of sebum is to provide the skin and the hair with a hydrophobic coating through the sebaceous ducts. Furthermore, human sebum has unique characteristics compared with that of other mammals. In particular, it contains a greatly reduced portion of cholesterol derivatives and a large quantity of squalene.

The importance of sebum in the homeostasis of the cutaneous tissue is known: the parts of the human body deficient in sebum (soles of the feet and palms of the hands) reveal a skin surface aesthetic quality and morphology that differ greatly from those of the rest of the body. Similarly, in the skin care field, it is apparent that certain unsightly vexations (dry skins) and important dermatological disorders (premature photoaging) may be attributed to the absence or insufficiency of sebum on the cutaneous surface. However, sebum in certain cases (major photooxidative stress, overproliferation of the bacterial flora, etc.) is probably a disrupting factor with regard to cutaneous homeostasis whose effects possibly result in undesirable secondary effects in the deep layers of the epidermis. Sebum is particularly present on the face and in the perinasal and forehead areas.

So it is necessary and always worthwhile to be able to have available a cosmetic composition that mimics sebum in order to be able to treat the consequences of a deficiency of natural sebum.

Furthermore, in the field of skin equivalents, to date no model includes a sebaceous gland equivalent and consequently all the existing models are totally lacking in sebum.

Here again it is worthwhile to be able to have available a cosmetic composition that mimics sebum in order to be able to apply it on a skin equivalent and thus obtain a skin equivalent comprising sebum, a skin equivalent that will be even closer to normal skin than the equivalents currently available.

It is to respond to these needs inter alia that the applicant has developed a cosmetic composition that mimics sebum.

Thus, the subject of the invention is a cosmetic composition that mimics sebum, comprising at least one lipophilic fraction in turn comprising at least:
from 5% to 20% of squalene,
from 50% to 70% of a mixture of triglycerides of linear fatty acids having a chain of 12 to 22 carbon atoms and of linear fatty acids having a chain of 12 to 22 carbon atoms,
from 15% to 25% of esters of linear fatty acids and of linear fatty alcohols having chains of 12 to 22 carbon atoms,
from 0.5% to 3% of esters of cholesterol whose acid fraction comprises a chain of 12 to 22 carbon atoms, and
from 0% to 5% of cholesterol,
   it being understood that the ratio of unsaturated fatty chains to saturated fatty chains lies between 10 and 0.1.

As linear fatty acid functions which are free or in the form of esters, which are saturated or unsaturated and which have a chain of 12 to 22 carbon atoms, lauric, myristic, palmitic, stearic, arachidic, docosanoic, myristoleic, palmitoleic, oleic, linoleic, linolenic, γ-linolenic or arachidonic acids can be cited.

As linear fatty alcohols which are saturated or unsaturated and which have a chain of 12 to 22 carbon atoms, lauryl, myristyl, cetyl, stearyl alcohols or oleyl alcohol can be cited.

According to a particular form of the invention, the squalene may be in a quantity representing from 12% to 18% of the lipophilic fraction.

According to another particular form of the invention, the mixture of triglycerides of linear fatty acids having a chain of 12 to 22 carbon atoms and of linear fatty acids having a chain of 12 to 22 carbon atoms may be in a quantity representing from 56% to 64% of the lipophilic fraction.

According to yet another form of the invention, the esters of linear fatty acids and of linear fatty alcohols having chains of 12 to 22 carbon atoms may be in a quantity representing from 19% to 22% of the lipophilic fraction.

Still according to the invention, the esters of cholesterol whose acid fraction comprises a chain of 12 to 22 carbon atoms may be in a quantity representing from 0% to 1.5% of the lipophilic fraction.

Finally, according to the invention, the cholesterol may be in a quantity representing from 1% to 3% of the lipophilic fraction.

Preferably, according to the invention, the ratio of unsaturated fatty chains to saturated fatty chains is between 5 and 0.2.

More particularly, the subject of the invention is a cosmetic composition that mimics sebum, characterized in that it comprises at least from 12% to 18% of squalene, from 0% to 10% of trimyristin, from 0% to 10% of trimyristolein, from 0% to 10% of tripalmitin, from 0% to 10% of tripalmitolein, from 0% to 20% of triolein, from 0% to 10% of tristearin, from 0% to 60% of glycerol, from 0% to 15% of oleic acid, from 0% to 10% of palmitic acid, from 0% to 15% of palmitoleic acid, from 0% to 10% of myristic acid, from 0% to 10% of myristoleic acid, from 5% to 20% of myristyl oleate, from 5% to 10% of palmityl oleate, from 0.5% to 3% of cholesteryl palmitate and from 0% to 5% of cholesterol.

This lipid composition will be such that the sum of the percentages of the free fatty acid fractions added to the sum of the percentages of the triglyceride fractions will be between 55% and 65%. In addition, the ratio of unsaturated fatty chains to saturated fatty chains is between 8 and 0.2.

A very particular composition of the invention is a composition which comprises at least 16% of squalene, 8% of tripalmitin, 18% of tripalmitolein, 12% of triolein, 10% of oleic acid, 7% of palmitoleic acid, 5% of myristoleic acid, 10% of myristyl oleate, 10% of palmityl oleate, 1% of cholesteryl oleate and 3% of cholesterol.

This cosmetic composition may contain all the active agents, in particular lipophilic active agents, liable to improve or modify the cutaneous condition. More particularly, the composition of the invention may comprise vitamin E at a concentration of between 0.0025% and 0.01% and preferably between 0.001% and 0.1%.

Another subject of the invention is a method of preparing a cosmetic composition as described, comprising a first step of weighing each of the ingredients included in the composition in a sterile receptacle, opaque to the light, previously coated with a non-stick coating, a second step of replacing the air with an inert gas, a third step of raising the temperature to a temperature between 50° C. and 100° C., preferably 80° C., and a fourth step of maintaining the temperature for a time between 25 and 45 minutes, preferably 30 and 40 minutes, with stirring.

Preferably, according to the invention, the non-stick coating may be obtained by applying a silane product, particularly a solution of dimethyldichlorosilane in 1,1,1-trichloroethane, very particularly of dimethyldichlorosilane at a concentration of between 10 g/l and 30 g/l, preferably at 20 g/l, in 1,1,1-trichloroethane.

According to the invention, the inert gas used in the preparation method may be chosen from argon, nitrogen, neon, krypton or xenon. Preferably, argon is used.

A further subject of the invention is a cosmetic composition liable to be obtained by the method that has just been described.

Another subject of the invention is the use of a cosmetic composition as described to alleviate the insightly vexations linked to a deficiency of sebum, particularly for treating dry skin.

The applicant has noted that, surprisingly, pretreatment with a composition whose fatty phase mimics the chemical and rheological properties of human sebum almost completely inhibited the effect of lateral diffusion of cosmetic formulations.

The transverse diffusion of the active agents and/or components of topically applied formulations through the human epidermis has been the subject of numerous studies in the past.

Less known, but nonetheless studied, is the ability of the superficial organization of the horny layer to generate a lateral diffusion of the molecules. The orders of magnitude (distance and speed) conventionally described for this phenomenon are however extremely small.

Recent work has called into question the limited character of this lateral diffusion and shown, in the case of clobetasol, that considerable quantities of active agents could be found, fairly quickly, at macroscopic distances from the point of application.

For those skilled in the art, this is a phenomenon which may be inconvenient if the desire is to target an active agent (forehead wrinkles and crow's feet, treatment of pigment blemishes, targeted body care of the slimming type, high capacity sunscreen in the highly exposed areas) for maximum effectiveness. To solve this problem, no sound solution has been recorded.

A sound solution means a solution that could be used to eliminate the phenomenon of lateral diffusion without having to systematically verify through a clinical test the lateral diffusion of the constituents of the formulation that is intended for the market and to be obliged to dramatically modify the composition of this formulation if this effect is observed.

From the foregoing, it is clear that a simple solution, easy to apply, of avoiding the effect of lateral diffusion would be highly desirable.

Now the applicant has been able to show that the cosmetic composition that mimics sebum of the invention has the effect of limiting the lateral diffusion of the cosmetic active agents or formulations.

Thus, the invention also relates to the use of a cosmetic composition that mimics sebum, particularly the one previously described, to control the lateral diffusion of the cosmetic active agents or formulations.

Preferably, the cosmetic composition that mimics sebum is applied prior to the application of the cosmetic active agents or formulations, in particular on non-sebum-generating (body) or sebum-generating but cleaned areas as is the case for example before putting make-up on the face. In this particular case, the invention will make it possible to avoid the lateral diffusion of the active agents and pigment contained in the make-up products.

So a further subject of the invention is a cosmetic method for limiting the lateral diffusion of the cosmetic formulations, characterized in that it comprises a step consisting in applying, to the skin, the mucous membranes or the scalp, a composition that mimics sebum as previously defined and a second step consisting in applying the cosmetic formulation to the area previously treated by the composition that mimics sebum.

The cosmetic formulation may in particular be a cosmetic active agent intended to improve the appearance or the condition of the skin, or a make-up product intended to color or mask portions of the cutaneous covering.

As has been seen above, the available skin equivalents are characterized by the absence of sebum or sebum equivalent. It would be particularly worthwhile, in order to study the phenomena of sebum generation and in particular of comedo generation, to have available a reconstructed skin model surfaced with a lipid composition similar to sebum.

Another subject of the invention is the use of a cosmetic composition that mimics sebum, particularly the one previously described, for the preparation of a skin equivalent comprising a sebum equivalent.

Specifically, unexpectedly, the applicant has found that it is possible to apply compositions that mimic sebum to reconstructed skins to obtain physiological or supra-physiological mimetic levels of sebum.

Advantageously, the sebum is applied in the form of a composition obtained by dispersion or of an emulsion of a sebum counterpart as previously defined in a physiological liquid, with a dilution of between 1/50 and 3/20, preferably approximately 1/20; this dilution being adapted by those skilled in the art according to the desired end result. Specifically, such formulations may be obtained without the addition of the formulation additives that are conventional in cosmetics, such as surfactants or preservatives. So the subject of the invention is the method of preparing compositions intended to be applied to skin equivalents.

Physiological liquid means in particular aqueous phases compatible with the tissues, such as water, saline solutions diluted to a greater or lesser degree, such as physiological saline, cell maintenance and culture media, serum, sweat and their mixtures in all proportions.

The composition obtained by dilution of the sebum counterpart is applied to the reconstructed skin, this application being able to be repeated regularly over time.

A further subject of the invention is a skin equivalent comprising a sebum equivalent, particularly a sebum equivalent consisting of the cosmetic composition of the invention.

More particularly, its subject is a skin equivalent comprising on its surface a sebum equivalent at a concentration of between 0 and 600 μg/cm², preferably 50 μg/cm² or more.

The final concentration will be adjusted by those skilled in the art according to the use of the reconstructed epidermis; it will preferably be 400 μg/cm² or less, particularly 300 μg/cm² or less.

The invention also relates to the use of a skin equivalent comprising a sebum equivalent as previously described to study the interactions between the cutaneous tissue, particularly the epidermis and/or the dermis, and the sebum, particularly in cutaneous homeostasis.

The invention also relates to the use of a skin equivalent comprising a sebum equivalent as previously described to study the interactions between the cutaneous tissue, particularly the epidermis and/or the dermis, and the sebum after modification of the latter by different exogenous and/or endogenous environmental factors or also to evaluate the effectiveness, the penetration or the toxicity of starting materials and of cosmetic formulations.

The applicant has noted surprisingly that the repeated treatment of a composition that mimics sebum rich in fatty acids on a reconstructed epidermis model has the effect of progressively thickening the superficial layers of the epidermis to the detriment of the deep living layers. The applicant has been able to demonstrate by measurements of cell proliferation and viability that the observed effect is not due to cytotoxicity.

Now in the presence of high bacterial activity, the triglycerides of sebum are transformed into free fatty acids, known for their comedogenic properties. Under these conditions, a hyperproliferation of the keratinocytes of the infrainfundibulum associated with a hypercornification is observed in the pilosebaceous duct. The applicant notes an analogy between the natural phenomenon of comedogenesis and that induced on a skin equivalent comprising a sebum equivalent rich in free fatty acids.

The applicant proposes therefore the use of such a biomimetic sebum, enriched in free fatty acids, for the preparation of a reconstructed skin model which makes it possible in particular to evaluate new anti-comedogenic substances or substance combinations.

Preferably, in these applications, the percentage of free fatty acids present in the composition that mimics sebum is 40% or more.

A further subject of the invention is the use of a skin equivalent comprising a sebum equivalent as previously described to study comedogenesis in the presence or absence of cosmetic active agents or formulations.

Finally, the object of the invention is a method of preparing a dispersion of the cosmetic composition of the invention, allowing its application both to normal skin and to skin equivalents, which comprises a step of dispersing the composition as prepared according to the invention and maintained at a temperature between 50° C. and 100° C., preferably 80° C., in a liquid physiological medium, preferably chosen from sweat, physiological saline, a physiological medium containing bovine serum albumin (BSA), skimmed milk or any other surfactant or alternatively a cell culture medium.

A preferred embodiment of the dispersion according to the invention consists in weighing, in a flask opaque to the light previously coated with a non-stick coating, a desired quantity of artificial sebum, adding to it a desired quantity of liquid physiological medium, heating at a temperature between 50° C. and 100° C., preferably at 80° C., for a time of between 10 seconds and 1 minute, preferably 20 seconds, placing the flask in an ultrasonic bath for a time of between 10 seconds and 2 minutes, preferably 30 seconds, and then stirring with a vortex stirrer for a time of between 5 seconds and one minute, preferably 10 seconds.

Preferably, according to the invention, the desired quantities of artificial sebum and of liquid physiological medium are such that the application of 2 μl of the dispersion provides the skin with the necessary physiological quantity, that is 100 μg of sebum.

Specifically, one of the advantages of the invention is that it allows the delivery onto the skin of a physiological quantity of sebum, that is to say, for a normal skin, between 100 and 200 μg/cm², which, to the knowledge of the applicant, is not achieved in the solutions provided by the prior art.

The following examples illustrate the invention while in no way limiting it. In these examples, reference will be made to the appended figure, representing the influence of the quality of the sebum on an Episkin model.

### EXAMPLE 1

### Sebum Formulation

The following cosmetic composition that mimics sebum S1 is produced. The quantities are given as percentages of the total weight of the composition:

| | |
|---|---|
| squalene | 16% |
| tripalmitin | 8% |
| tripalmitolein | 18% |
| triolein | 12% |
| oleic acid | 10% |
| palmitoleic acid | 7% |
| myristoleic acid | 5% |
| myristyl oleate | 10% |
| palmityl oleate | 10% |
| cholesteryl oleate | 1% |
| cholesterol | 3% |

In a screw-top amber glass flask, previously silanized (Repel Silane, dimethyldichlorosilane solution, 20 g/l in 1,1,1-trichloroethane, Pharmacia Biotech No. 17-1331-01) and autoclaved, weigh the various ingredients involved in the composition of the sebum S1, add a magnetic bar and fill the flask with an inert gas of argon type to prevent any peroxidation of the ingredients in the air. Place the sebum in a heating bath at 80° C. and with stirring for 30 to 40 minutes until a perfectly clear liquid is obtained.

Precautions:

The reconstituted sebum solidifies after 10 minutes at ambient temperature, so it is necessary to keep it in a water bath at 80° C. until the preparation of the dispersion.

The sebum is stored under argon.

### EXAMPLE 2

### Preparation of the Dispersion

In a previously silanized amber flask, weigh 10 mg of artificial sebum S1 of example 1, then add 200 μl of culture medium DMEM/F12 1:1 (Dulbecco's modified Eagle medium/nutrient mixture-F12). The flask is then heated in the water bath at 80° C. for 20 seconds and then placed in an ultrasonic bath for 30 seconds and finally stirred with a vortex stirrer for 10 seconds.

The desired quantity of artificial sebum S1 may then be applied to the skin (normal or artificial) by spreading with a spatula.

The dispersed form has the advantage of allowing the application of a physiological quantity of artificial sebum, that is between 100 and 200 μg/cm².

### EXAMPLE 3

### Effect of Lateral Diffusion for Two Cosmetic Formulations

The effect of diffusion of the formulations is estimated on the forearms of volunteer subjects. The formulations are applied at the rate of 2 mg.cm⁻¹. The surface area of application is a rectangle 4 cm by 7 cm (28 cm² are therefore treated) whose length is positioned in the axis of the arm. After one hour, the tape stripping method (Wigman, H. et al., (1999) Skin Pharmacopée. Apple. Skin Physiol. 12, 46-53.5) is used to evaluate the residual quantity of butylmethoxy-dibenzoylmethane in the central part and the part having migrated more than 3 mm from the edges of the rectangle.

Result: on the treated forearms, an average of 82% of the butylmethoxydibenzoylmethane applied is found in the central part for formulation 1 and 69% for formulation II. In parallel, the quantity of butylmethoxydibenzoylmethane that can be taken from the upper or lower end of the rectangle (relative to the arm) over surface areas of 1.9 cm×4 cm placed at 3 mm from the application area is 0.8% for formulation 1 and 7.6% for formulation II.

Compositions of formulations I and II:

The quantities are given as percentages by weight of the total weight of the composition

Formulations

| | I | II |
|---|---|---|
| PEG-100 Stearate | 0.75 | 0.75 |
| Glyceryl Stearate | 0.75 | 0.75 |
| Palmitic Acid | 1.21 | 1.21 |
| Myristic Acid | 0.0825 | 0.0825 |
| Stearic Acid | 1.4575 | 1.4575 |
| Cetyl Alcohol | 0.5 | 0.5 |
| Dimethicone | 0.5 | 0.5 |
| C12-15 Alkyl Benzoate | 8.5 | 20.0 |
| Butylmethoxydibenzoylmethane | 3.72 | 3.72 |
| Methylparaben | 0.174 | 0.174 |
| Butylparaben | 0.042 | 0.042 |
| Propylparaben | 0.021 | 0.021 |
| Isobutylparaben | 0.021 | 0.021 |
| Phenoxyethanol | 0.7 | 0.7 |
| Ethylparaben | 0.042 | 0.042 |
| Triethanolamine | 0.45 | 0.45 |
| Glycerol | 5.0 | 5.0 |
| Potassium Cetyl Phosphate | 1.0 | 1.0 |
| Carbomer | 0.3 | 0.3 |
| Octocrylene | 10.0 | — |
| Water | q.s. for | q.s. for |
| | 100% | 100% |

### EXAMPLE 4

### Effect of the Pretreatment by the Sebum S1 of Example 1 on the Lateral Diffusion of Formulation II

The protocol of example 3 is repeated with formulation II. 1 hour before the application of formulation II, a 5% suspension of sebum S1 of example 1 in a 0.9% aqueous NaCl solution is applied to the right forearm (the left forearm being used as a control), such that a measurement with the sebumeter (MONADERM™) indicates an application of 200 μg.cm⁻². The surface area of application of the sebum S1 is a rectangle whose centre is identical to that of the application of the formulations (example 3) and whose length and width are 2 cm greater than this rectangle (that is, a rectangle of 6×7 cm).

Results: under these conditions, on the forearm pretreated with the sebum S1, the quantity of butylmethoxydibenzoylmethane found in the central part is 94% (compared with 69% without pretreatment) and the quantity of butylmethoxydibenzoylmethane that can be taken from the top or bottom end of the rectangle (relative to the arm) over surface areas of 1.9 cm×4 cm placed at 3 mm from the application area is reduced to 0.7% (compared with 7.6%).

It will be noted that the quantity of sebum S1 really applied in this example (≈200 μg. cm⁻²) is analogous to the quantity of sebum naturally found on the surface of the faces (anatomical area, naturally seborrheic) of individuals with normally greasy skin. This however does not mean that the individuals with greasy skin would not be affected by the phenomenon of lateral diffusion because face care products are usually applied to a clean face and hence free from lipids.

### EXAMPLE 5

### Influence of the Quality of the Sebum on a Reconstructed Skin Model

Compositions progressively enriched with free fatty acids are applied on an Episkin® reconstructed skin model in order to obtain a model of comedogenesis. The appended figure shows the modifications in the proliferation/differentiation balance on day 13. These photographs illustrate the differences in morphology presented by a reconstructed skin model (Episkin®) progressively enriched with free fatty acids (from 0% of fatty acids (far left photograph) to 60% of free fatty acids (far right photograph)) and progressively depleted in triglycerides (from 60% to 0%). The sebum rich in triglycerides favors the maintenance and appearance of the granular layer, the sebum rich in fatty acids causes the thickening of the stratum corneum.

The repeated treatment of a composition that mimics sebum rich in fatty acids has the effect of progressively thickening the superficial layers of the epidermis to the detriment of the deep living layers. The thickening of the epidermis is observed starting from a sebum composition comprising 40% of fatty acids. This change in morphology induced in vitro has an analogy with the natural phenomenon of comedogenesis. In vivo, a hyperproliferation of the keratinocytes of the infrainfundibulum associated with a hypercornification is observed in the pilosebaceous duct.

1. A cosmetic composition that mimics sebum, comprising at least one lipophilic fraction in turn comprising at least:
from 5% to 20% of squalene,
from 50% to 70% of a mixture of triglycerides of linear fatty acids having a chain of 12 to 22 carbon atoms and of linear fatty acids having a chain of 12 to 22 carbon atoms,
from 15% to 25% of esters of linear fatty acids and of linear fatty alcohols having chains of 12 to 22 carbon atoms,
from 0.5% to 3% of esters of cholesterol whose acid fraction comprises a chain of 12 to 22 carbon atoms, and
from 0% to 5% of cholesterol,
it being understood that the ratio of unsaturated fatty chains to saturated fatty chains lies between 10 and 0.1.

2. The composition as claimed in claim 1, which the squalene is in a quantity representing from 12% to 18% of the lipophilic fraction.

3. The composition as claimed in claim 1, wherein the mixture of triglycerides of linear fatty acids having a chain of 12 to 22 carbon atoms and of linear fatty acids having a chain of 12 to 22 carbon atoms are in a quantity representing from 56% to 64% of the lipophilic fraction.

4. The composition as claimed in any one of claims claim 1, wherein the esters of linear fatty acids and of linear fatty alcohols having chains of 12 to 22 carbon atoms are in a quantity representing from 19% to 22% of the lipophilic fraction.

5. The composition as claimed in 1 to 4, claim 1, wherein the esters of cholesterol whose acid fraction comprises a chain of 12 to 22 carbon atoms are in a quantity representing from 0% to 1.5% of the lipophilic fraction.

6. The composition as claimed in claim 1, wherein the cholesterol is in quantity representing from 1% to 3% of the lipophilic fraction.

7. The composition as claimed in claim 1, characterized in that wherein the ratio of unsaturated fatty chains to saturated fatty chains is between 5% to 0.2.

8. A cosmetic composition that mimics sebum, which comprises at least from 12% to 18% of squalene, from 0% to 10% of trimyristin, from 0% to 10% of trimyristolein, from 0% to 10% of tripalmitin, from 0% to 10% of tripalmitolein, from 0% to 20% of triolein, from 0% to 60% of glycerol, from 0% to 10% of tristearin, from 0% to 15% of oleic acid, from 0% to 10% of palmitic acid, from 0% to 15% of palmitoleic acid, from 0% to 10% of myristic acid, from 0% to 10% of myristoleic acid, from 5% to 20% of myristyl oleate, from 5% to 10% of palmityl oleate, from 0.5% to 3% of cholesteryl palmitate and from 0% to 5% of cholesterol, it being understood that the sum of the percentages of the free fatty acid fractions added to the sum of the percentages of the triglyceride fractions will be between 55% and 65% and that the ratio of unsaturated fatty chains to saturated fatty chains is between 8 and 0.2.

9. A cosmetic composition that mimics sebum, which comprises at least 16% of squalene, 8% of tripalmitin, 18% of tripalmitolein, 12% of triolein, 10% of oleic acid, 7% of palmitoleic acid, 5% of myristoleic acid, 10% of myristyl oleate, 10% of palmityl oleate, 1% of cholesteryl oleate and 3% of cholesterol.

10. The cosmetic composition that mimics sebum as claimed in claim 1, which also comprises vitamin E at a concentration of between 0.0025% and 0.01% and preferably between 0.001% and 0.1%.

11. A method of preparing a cosmetic composition as described in claim 1, comprising a first step of weighing each of the ingredients included in the composition in a sterile receptacle, opaque to the light, previously coated with a non-stick coating, a second step of replacing the air with an inert gas, a third step of raising the temperature to a temperature between 50° C. and 100° C., and a fourth step of maintaining the temperature for a time of between 25 and 45 minutes, preferably 30 and 40 minutes, with stirring.

12. The method as claimed in claim 11, the inert gas being chosen from argon, nitrogen, neon, krypton or xenon.

13. The method as claimed in claim 12, wherein the inert gas is argon.

14. The cosmetic composition obtained by the method of claim 11.

15. A method to alleviate the unsightly vexations linked to a deficiency of sebum comprising using the composition according to claim 1.

16. A method for treating dry skin comprising using the composition according to claim 1.

17. A method to control the lateral diffusion of cosmetic active agents or formulations comprising using the composition according to claim 1.

18. The method as claimed in claim 17, wherein the composition is applied prior to the application of the cosmetic active agents or formulations.

19. A method for the preparation of a skin equivalent comprising a sebum equivalent comprising using the composition according to claim 1.

20. A skin equivalent comprising a sebum equivalent.

21. The skin equivalent as claimed in the preceding claim 20, wherein the sebum equivalent is a cosmetic composition.

22. A method to study the interactions between the sebum, the epidermis and/or the dermis, particularly in cutaneous homeostasis comprising using the composition according to claim 1.

23. A method to study the interactions between the cutaneous tissue, particularly the epidermis and/or the dermis, and the sebum after modification of the latter by different exogenous and/or endogenous environmental factors comprising using the composition according to claim 1.

24. A method to evaluate the effectiveness, the penetration or the toxicity of starting materials and of cosmetic formulations comprising using the composition according to claim 1.

25. A method to study comedogenesis in the presence or absence of cosmetic active agents or formulations comprising using the composition according to claim 1.

26. A method of preparing a dispersion of the cosmetic composition of the invention comprising a step of dispersing the composition as prepared according to the invention and its maintenance at a temperature between 50° C. and 100° C., in a liquid physiological medium.

27. The method as claimed in claim 26, wherein the liquid physiological medium is chosen from sweat, physiological saline, or alternatively a cell culture medium.

28. The method as claimed in claim 2, wherein the desired quantities of artificial sebum and of liquid physiological medium are such that the application of 2 μl of the dispersion provides the skin with the necessary physiological quantity, that is 100 μg of sebum.
